(19) United States
(12) Patent Application Publication
(10) Pub. No.: US 2006/0205808 A1
(43) Pub. Date: Sep. 14, 2006

(54) Anti-inflammatory and cure for ageing, Alzheimer's disease on phloroglucinol derivatives

(75) Inventors: Chun-Nan Lin, San Ming District Kaohsiung City (TW); Jih-Pyang Wang, San Ming District Kaohsiung City (TW); Jing-Ru Weng, San Ming District Kaohsiung City (TW)

Correspondence Address: DYKAS, SHAVER & NIPPER, LLP, P.O. BOX 877, BOISE, ID, 83701-0877, US

(73) Assignee: Kaohsiung Medical University, Kaohsiung City (TW)

(21) Appl. No.: 11/078,194

(22) Filed: Mar. 10, 2005

(51) Int. Cl. A61K 31/343; C07C 49/105; C07D 307/92; A61K 31/12

(52) U.S. Cl. 514/468; 514/690; 549/458; 568/373

(57) ABSTRACT

The fresh seeds obtained from the fresh fruits of Garcinia subelliptica, were extracted with chloroform at room temperature. The CHCl₃ extract was concentrated under reduced pressure to afford a brown residue. This residue was subjected to column chromatography (silica gel) and eluted with appropriate solvent system to give two phloroglucinol derivatives (Compounds 1 and 4). Compound 1 exhibited potent inhibitory effects on the release of β-glucuronidase and lysozyme from rat neutrophils stimulated with formyl-Met-Leu-Phe(fMLP)/cyto-chalasin B (CB). Compound 1 also exhibited potent inhibitory effect on the superoxide anion generation in rat neutrophils stimulated with fMLP/CB. Compound 4 exhibited potent inhibitory effect on NO production in lipopolysaccharide (LPS)/interferon-γ (IFN-γ)-activation in N9 cells. These Compounds may be developed as anti-inflammatory, anti-cancer agent, cure for ageing, and Alzheimer's disease treatment drugs.

### BACKGROUND OF THE INVENTION

### Field of the Invention

This application describes the method of preparing two novel phloroglucinol derivatives from the fresh seeds of Garcinia subelliptica and the use of these phloroglucinol derivatives as an anti-inflammatory, as an anti-cancer agent, as a cure for the effects of ageing, and/or as an Alzheimer's disease cure.

Garcinia subelliptica Merr. (Guttiferae) typically grown as a potted or garden plant is rich in variety of prenyl xanthones and oxygenated xanthone, as reported in Iinuma, M. et al. (Phytochemistry, 35, pp. 1355-1360) and Minami, H. et al. (Phytochemistry, 36, pp. 501-506).

We have previously reported (Chem. Commun., pp. 1315-1316 (1996) and J. Nat. Prod., 61, pp. 1015-1016 (1998)) various constituents and antioxidant xanthones from the wood and root bark of Garcinia subelliptica Merr. (Guttiferae). In the search for bioactive constituents in Formosan Guttiferae plants, the bioactive constituents of the seeds of G. subelliptica were investigated and three novel triterpenoids and four novel phloroglucinol derivatives were reported.

A continuing study (Chem. Eur. J. 2003, 9, 5520-5527) of the constituents of the seeds of G. subelliptica yielded three novel phloroglucinol derivatives, namely garcinielliptones F (FIG. 1), H (FIG. 3), and I (FIG. 4), and two novel terpenoids, namely garcinielliptones G (FIG. 2) and J (FIG. 5), with a new skeleton. Garsubellin A and garcinielliptin oxide, isolated from the seeds of this plant, strongly inhibited the chemical mediators release from mast cells and neutrophils. The structure elucidation of the five novel compounds and the anti-inflammatory effects of 1-5 are reported.

Additional objects, advantages and novel features of the invention will be set forth in part in the description which follows and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

The present invention relates to the preparation methods of two novel phloroglucinol derivatives from the fresh seeds of Garcinia subelliptica and the pharmacological uses of said phloroglucinol derivatives as an anti-inflammatory, as an anti-cancer agent, as a cure for the effects of ageing, and/or as a cure for Alzheimer's disease.

The purpose of the foregoing Abstract is to enable the United States Patent and Trademark Office and the public generally, and especially the scientists, engineers, and practitioners in the art who are not familiar with patent or legal terms or phraseology, to determine quickly from a cursory inspection, the nature and essence of the technical disclosure of the application. The Abstract is neither intended to define the invention of the application, which is measured by the claims, nor is it intended to be limiting as to the scope of the invention in any way.

Still other objects and advantages of the present invention will become readily apparent to those skilled in this art from the following detailed description wherein I have shown and described only the preferred embodiment of the invention, simply by way of illustration of the best mode contemplated by carrying out my invention. As will be realized, the invention is capable of modification in various obvious respects all without departing from the invention. Accordingly, the drawings and description of the preferred embodiment are to be regarded as illustrative in nature, and not as restrictive in nature.

### BRIEF DESCRIPTION OF THE DRAWINGS

FIG. 1 shows the structure of phloroglucinol derivative garcinielliptone F (Compound 1).

FIG. 2 shows the structure of terpenoid garcinielliptone G (Compound 2).

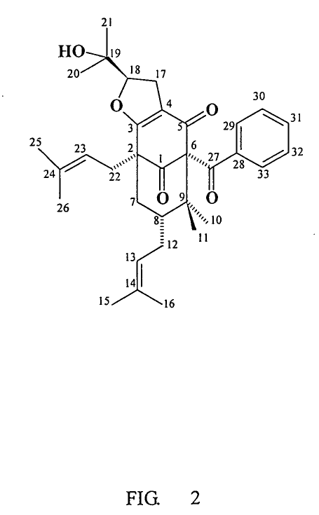

FIG. 3 shows the structure of phloroglucinol derivative garcinielliptone H (Compound 3).

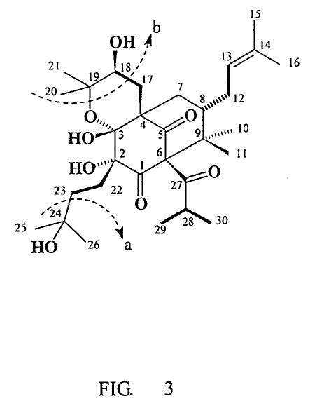

FIG. 4 shows the structure of phloroglucinol derivative garcinielliptone I (Compound 4).

FIG. 5 shows the structure of terpenoid garcinielliptone J (Compound 5).

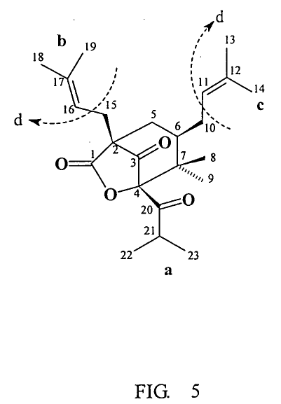

FIG. 6 (Scheme 1) shows the preparation methods from the fresh seeds of Garcinia subelliptica.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

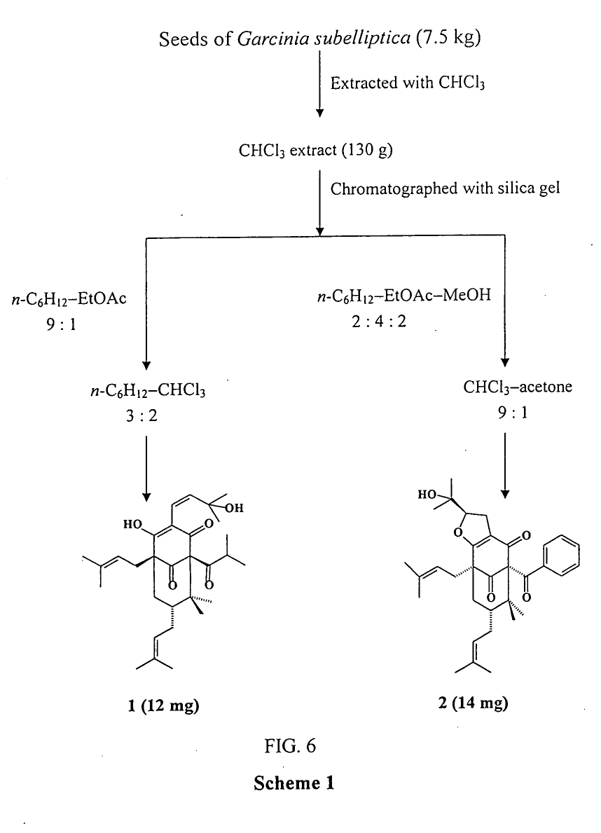

In the preferred embodiment of the present invention, the fresh seeds obtained from the fresh fruits of Garcinia subelliptica were extracted with chloroform at room temperature. These fruits of G. subelliptica were collected at Kaohsiung, Taiwan. The CHCl₃ extract was concentrated under reduced pressure to obtain a brown residue. This residue was subjected to column chromatography (silica gel) and eluted with an appropriate solvent system (FIG. 6-Scheme 1) to give novel phloroglucinol and terpenoid derivatives. That appropriate solvent system including some organic solvents, such as methanol, n-hexane (n-C₆H₁₂), ethyl acetate (EtOAc), acetone and chloroform (CHCl₃). For eluted suitable compounds, two or three organic solvents are mixed together, such as n-hexane/CHCl₃ (3:2), n-hexane/ethyl acetate (9:1), n-hexane/acetone (12:1), n-C₆H₁₂/EtOAc/MeOH (2:4:2). After isolation, the atomic analysis, optical rotations, spectrums of mass, infrared, tritium magnetic resonance (¹H-NMR, CDCl₃) and UV were then determined for the purified products. Suitable experimental models were used to review the data. Structure of this novel phloroglucinol and terpenoid were revealed by these physical and chemical data.

The processes of degranulation of mast cells and neutrophils contributes to inflammatory disorders, combining potent inhibition of chemical mediators released from mast cells and neutrophil degranulation, would suggest a promising anti-inflammatory agent.

Macrophages are important in nonspecific host resistance to microbial pathogens and serve as central regulators of specific immune response. Upon activation, nitric oxide (NO) accompanied with other chemical mediators is released in response to bacterial endotoxin (lipopolysaccharide, LPS). NO plays a central role in macrophage-induced cytotoxicity, however the excess of NO may contribute to the pathophysiology of septic shock. Microglial cell, the brain resident macrophages, have detrimental as well as beneficial effects on the surrounding cells and are believed to be involved in most inflammatory, infectious, and degenerative diseases of the central nervous system (CNS). NO released by activated microglial cells in response to LPS are likely to have a crucial role in mediating the interaction between microglial and other cells present in the nervous system, as they are known to regulate inflammation, immune functions, blood vessel dilation, neurotransmission, and neural cells survival.

Hence, the inhibition of NO released from microglial cells or macrophages is also a rational therapeutic approach to treat a variety of inflammatory, neuronal observed in ageing and Alzheimer's disease, and cancer.

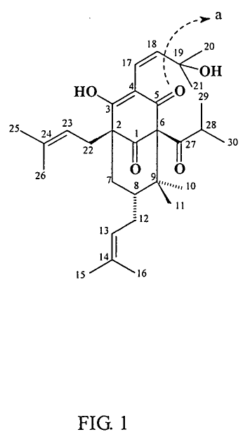

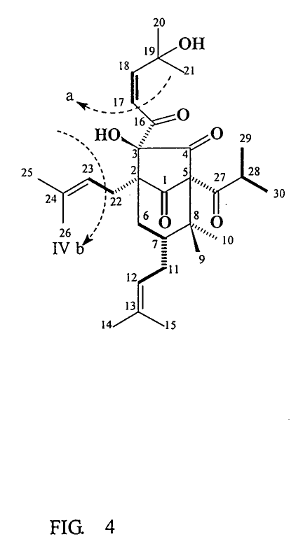

Compound 1 (FIG. 1) exhibited potent inhibitory effects on the release of β-glucuronidase and lysozyme from rat neutrophils stimulated with fMLP (1 μM)/CB (5 μg mL⁻¹). (Table 1). Compound 1 also exhibited potent inhibitory effect on the superoxide anion generation in rat neutrophils stimulated with fMLP (03 μM)/CB (5 μg mL⁻¹) (Table 2), and indicated the involvement of PMA-independent signaling pathway. Compound 4 (FIG. 4) exhibited potent inhibitory effect on NO production in LPS (10 ng mL⁻¹)/IFN-γ (10 U mL⁻¹)/interferon-γ (IFN-γ)-activation in N9 cells (Table 3).

Thus, it appears that phloroglucinol derivatives inhibit the chemical mediator release from inflammatory cells. Compound 1 showed potent inhibitory effects on the release of β-glucuronidase, lysozyme, and superoxide anion from neutrophils, which suggests that Compound 1 may be valuable in the therapeutic treatment or prevention of peripheral diseases associated with the excess release of β-glucuronidase, lysozyme, and superoxide anion from neutrophils. The inhibition of NO by Compound 4 in microglial cells may be of value in the therapeutic treatment or prevention of certain central as well as peripheral inflammatory diseases and cancer associated with the increase of NO production.

The anti-inflammatory activities of Compounds 1-5 were studied in vitro by measuring the inhibitory effects on the chemical mediator release from mast cells, neutrophils, macrophages, and microglial cells. Compounds 1-5 did not cause a significant inhibition of mast cell degranulation stimulated with compound 48/80 (10 μg mL⁻¹) (data not shown). Mepacrine was used in this experiment as a positive control.

FMLP (0.3 μM)/CB (5 μg mL⁻¹) or phorbol myristate acetate (PMA) (3 nM) stimulated superoxide anion generation in rat neutrophils. As shown in Table 2, Compound 1 had a potent inhibitory effect on fMLP/CB-induced superoxide anion generation in rat neutrophils in a concentration-dependent manner with an IC₅₀ value of 17.0±0.9 μM, while 2-5 had no significant effects. The observation that Compounds 1-5 had no appreciable effect on PMA-induced response suggests the involvement of a PMA-independent signaling pathway. Trifluoperazine was used in this experiment as a positive control.

Treatment of RAW 264.7 macrophage-like cells with LPS (1 μg mL⁻¹) or N9 microglial cells with LPS (10 ng mL⁻¹)/IFN-γ (10 U mL⁻¹) for 24 h induced NO production as assessed by measuring the accumulation of nitrite, a stable metabolite of NO, in the media, based on the Griess reaction. As shown in Table 3, the production of NO induced by LPS/IFN-γ in N9 cells was suppressed in a concentration dependent manner by Compound 4 with an IC₅₀ value of 7.4±0.2 μM, while 1-3, and Compound 5 did not show significant effects on the production of NO induced by LPS in RAW 264.7 cells or induced by LPS/IFN-γ in N9 cells. 1400 W was used in this experiment as a positive control.

The present study suggests that Compound 1 may have value in the therapeutic treatment or prevention of peripheral diseases associated with the release of β-glucuronidase, lysozyme, and superoxide anion from neutrophils, and the inhibition of NO production by Compound 4 in microglial cells may have value in the therapeutic treatment or prevention of certain central as well as peripherial inflammatory diseases associated with the increase of NO production.

Table 1 shows the inhibitory effects of Compounds 1 and 4 on the release of β-glucuronidase and lysozyme from rat neutrophils stimulated with fMLP (1 μM)/CB (5 μg mL⁻¹).

TABLE 1

IC₅₀ (μM)^{a}

| Compound | β-Glucuronidase | Lysozyme |
|---|---|---|
| 1 | 26.9 ± 2.6 | 20.0 ± 1.3 |
| 4 | >30 (15.6 ± 2.7) | >30 (−1.6 ± 7.3) |
| Trifluoperazine | 10.6 ± 0.9 | 13.2 ± 0.7 |

^{a}When 50% inhibition could not be reached at the highest concentration, the % of inhibition is given in parentheses.
Data are presented as means ± s.e.m. (n = 3-5).
Trifluoperazine was used as a positive control.

FIG. 8 (Table 2) shows the inhibitory effects of Compounds 1 and 4 on the superoxide anion in rat neutrophils stimulated with fMLP (0.3 μM)/CB (5 μg mL⁻¹) or PMA (3 nM).

TABLE 2

IC₅₀ (μM)^{a}

| Compound | fMLP/CB | PMA |
|---|---|---|
| 1 | 17.0 ± 0.9 | >30 (46.8 ± 4.5) |
| 4 | >30 (2.8 ± 5.7) | >30 (−76.3 ± 7.3) |
| Trifluoperazine | 6.6 ± 0.2 | 2.7 ± 0.6 |

^{a}When 50% inhibition could not be reached at the highest concentration, the % of inhibition is given in parentheses.
Data are presented as means ± s.e.m. (n = 3-5).
Trifluoperazine was used as a positive control.

Table 3 shows the inhibitory effects of Compounds 1 and 4 on the accumulation of NO₂⁻ in the culture media of RAW 264.7 cells in response to LPS (1 μg mL⁻¹) and N9 cells in response to LPS (10 ng mL⁻¹)/IFN-γ (10 U mL⁻¹).

TABLE 3

IC₅₀ (μM)^{a}

| Compound | RAW 264.7 cells | N9 cells |
|---|---|---|
| 1 | >10 (22.9 ± 2.9) | >3 (31.2 ± 0.7) |
| 4 | >30 (17.4 ± 1.5) | 7.4 ± 0.2 |
| 1400W^{b} | 3.0 ± 0.2 | 2.2 ± 0.1 |

^{a}When 50% inhibition could not be reached at the highest concentration, the % of inhibition is given in parentheses.
Data are presented as means ± s.e.m. (n = 3-5).
^{b}N-(3-Aminomethyl) benzylacetamidine (1400 W).

In summary, the present invention has the features of creativity, novelty and innovativity. Although the present invention uses just a few better preparation examples disclosed as above, its application will not be limited to them.

### EXAMPLE

Optical rotations: JASCO model DIP-370 digital polarimeter.

UV Spectra: JASCO UV-VIS Spectrophotometer; λₘₐₓ (log ε): in nm.

IR spectra: Hitachi 260-30 Spectrophotometer; \overline{ν} in cm⁻¹.

¹H- and ¹³C—NMR spectra: Varian Unity-400 spectrometer; 400 and 100 MHz, resp.; δ in ppm, J in Hz.

MS: JMS-HX100 mass spectrometer; m/z (rel. %).

Plant Material: The fruits of G. subelliptica were collected at Kaohsiung, Taiwan, during July 2001. A voucher specimen (2003) has been deposited at the Department of Medicinal Chemistry, School of Pharmacy, Kaohsiung Medical University.

Extraction and Isolation: The fresh seeds (7.5 kg) obtained from the fresh fruits (22.8 kg) of G. subelliptica, were extracted with chloroform at room temperature. The CHCl₃ extract was concentrated under reduced pressure to afford a brown residue (130 g). This residue was subjected to column chromatography (silica gel). Elution with n-hexane/CHCl₃ (3:2) yielded 1 (12 mg). Elution with n-hexane/acetone (12:1) yielded 2 (10 mg). Elution with CHCl₃/ethyl acetate (9:1) yielded 3 (16 mg) and 5 (18 mg). Elution with CHCl₃/acetone (9:1) yielded 4 (14 mg).

Garcinielliptone F (Compound 1/Formula I): colorless oil; [α]²⁵ _{D}=−23° (c 0.09, CHCl₃); IR (film on NaCl): \overline{ν}=3439 (OH), 1724 (C═O), 1639 cm⁻¹; UV (MeOH) λₘₐₓ (log ε): 265 (4.07) nm; ¹H NMR ([D₁]CHCl₃): δ=1.39 (m), 1.89 (dd, 13.2, 3.2), 1.44 (m), 1.00 (s), 1.22(s), α 1.67(m), β 2.09(m), 4.95 (t, 7.2), 1.64 (s), 1.65 (s), 6.47 (d, 10.0), 5.34 (d, 10.0), 1.39(s), 1.43(s), 2.41 (dd, 14.8, 8.0), 2.49 (dd, 14.8, 6.0), 5.00 (t, 7.2), 1.54 (s), 1.67(s), 2.07 (m), 1.11 (d, 6.4), 1.02 (d, 6.4); ¹³C NMR: 21.5 (C-10), 25.9 (C-16), 18.1 (C-26), 28.6 (C-20), 22.9 (C-11), 25.9 (C-15), 28.3 (C-21), 17.8 (C-25), 26.5 (C-12), 115.4 (C-17), 29.0 (C-22), 39.2 (C-7), 43.3 (C-8), 46.8 (C-9), 56.7 (C-2), 81.9 (C-19), 83.5 (C-6), 123.7 (C-18), 114.5 (C-4), 119.3 (C-23), 122.6 (C-13), 15.6 (C-30), 20.5 (C-29), 133.8 (C-14), 133.3 (C-24), 42.5 (C-28), 170.8 (C-3), 188.3 (C-5), 209.0 (C-27), 206.3 (C-1); CIMS: m/z (%): 530 [514+NH₃—H]⁻ (4), 514 [498+NH₃—H]⁻ (32), 498 [482+NH₃—H]⁻ (35), 483 [M-1]⁻ (25), 482 (47), 466 (100), 454 (4), 424 (5).

Garcinielliptone I (Compound 4/Formula II): colorless oil; [α]²⁵ D=−53° (c 0.14, CHCl₃); IR (film on NaCl): \overline{ν}=3454 (OH), 1767, 1727, 1708 (C═O), 1665, 1451 cm⁻¹; UV (MeOH) λₘₐₓ (log ε): 222 (4.18)nm; ¹H NMR ([D₁]CHCl₃): δ=1.13 (s, H₃-10), 1.22 (s, H₃-20), 1.32 (s, H₃-21), 1.41 (s, H₃-11), 1.50 (m, H_{β}-7), 1.58 (s, H₃-16), 1.66 (m, Hp-8), 1.69 (s, H₃-15), 1.69 (s, H₃-25), 1.72 (s, H₃-26), 1.76 (dd, J=10.8, 5.6 Hz, H_{α}-12), 2.01 (dd, J=13.2, 4.0 Hz, H_{β}-7), 2.19 (dd, J=10.8, 9.6 Hz, H_{β}-12), 2.53 (m, H₂-22), 2.93 (dd, J=15.2, 7.2 Hz, H_{α}-17), 3.05 (dd, J=15.2, 10.4 Hz, H_{β}-17), 4.85 (dd, J=10.4, 7.2 Hz, H-18), 4.98 (t, J=6.8 Hz, H-13), 5.04 (t, J=6.8 Hz, H-23), 7.25 (m, H₃-30), 7.25 (m, H₃-32), 7.40 (m, H₃-31), 7.55 (m, H-29), 7.55 (m, H₃-33); ¹³C NMR: 15.9 (C-10), 18.0 (C-16), 18.2 (C-26), 23.1 (C-20), 23.7 (C-11), 26.0 (C-15), 26.0 (C-21), 26.0 (C-25), 27.0 (C-12), 27.1 (C-17), 29.0 (C-22), 39.7 (C-7), 43.3 (C-8), 47.7 (C-9), 55.5 (C-2), 71.8 (C-19), 79.0 (C-6), 93.0 (C-18), 118.3 (C-4), 120.4 (C-23), 122.4 (C-13), 128.0 (C-30), 128.0 (C-32), 128.1 (C-29), 128.1 (C-33), 132.2 (C-31), 133.5 (C-14), 134.8 (C-24), 136.6 (C-28), 175.6 (C-3), 187.7 (C-5), 193.2 (C-27), 206.5 (C-1); EIMS: m/z (%): 498 [M-2]⁺ (6), 482 (10), 414 (33), 343 (100), 287 (44); HR-EIMS: found: 498.2996 [M-2]⁺, calcd for C₃₀H₄₂O₆⁺498.2981.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

I claim:

1. A compound containing the phloroglucinol moiety of the following:

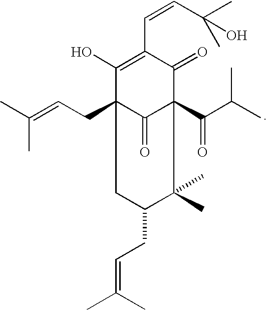

2. A composition comprising a pharmaceutical compound according to claim 1 which has anti-inflammatory and anti-allergic therapeutic utility, and using said compound as the main component.

3. A composition comprising a pharmaceutical compound according to claim 2 including various diluents and/or excipients.

4. A composition comprising a pharmaceutical compound according to claim 1 which has anti-inflammatory and cure for ageing therapeutic utility, and using said compound as the main component.

5. A composition comprising a pharmaceutical compound according to claim 4 including various diluents and/or excipients.

6. A compound containing the phloroglucinol moiety of the following:

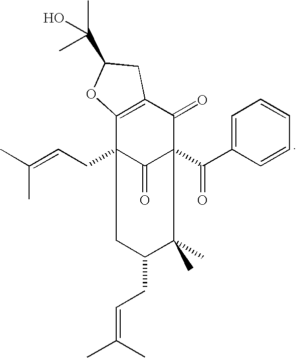

7. A composition comprising a pharmaceutical compound according to claim 6 which has anti-inflammatory and anti-allergic therapeutic utility, and using said compound as the main component.

8. A composition comprising a pharmaceutical compound according to claim 7 including various diluents and/or excipients.

9. A composition comprising a pharmaceutical compound according to claim 6 which has anti-inflammatory and cure for ageing therapeutic utility, and using said compound as the main component.

10. A composition comprising a pharmaceutical compound according to claim 9 including various diluents and/or excipients.

11. A composition comprising a pharmaceutical compound containing the phloroglucinol moiety selected from the group consisting of: 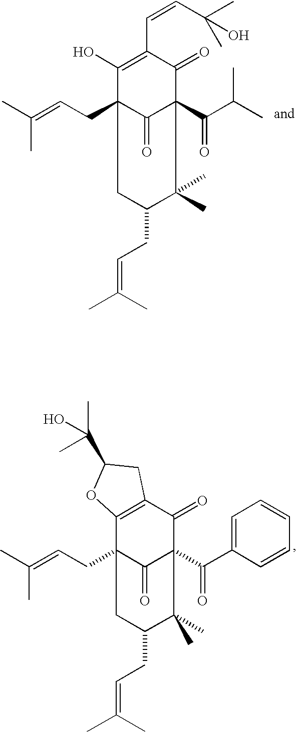
said composition having therapeutic utility with respect the treatment and/or prevention of peripheral diseases associated with the excess release of β-glucuronidase, lysozyme, and superoxide anion from neutrophils and/or diseases associated with an increase in nitric oxide production by activated microglial cells in response to LPS, and using said selected pharmaceutical compound as the main component.

12. A composition comprising a pharmaceutical compound according to claim 11 including various diluents and/or excipients.
